SEQUENCE LISTING (144 sequences)

SEQ ID NO: 1 - DNA, 15 bp, Artificial Sequence
  PNA probe Let-7a
tatacaacctactac

SEQ ID NO: 2 - DNA, 15 bp, Artificial Sequence
  PNA probe Let-7b
ccacacaacctacta

SEQ ID NO: 3 - DNA, 15 bp, Artificial Sequence
  PNA probe Let-7c
catacaacctactac

SEQ ID NO: 4 - DNA, 15 bp, Artificial Sequence
  PNA probe Let-7d
tatgcaacctactac

SEQ ID NO: 5 - DNA, 15 bp, Artificial Sequence
  PNA probe Let-7e
tatacaacctcctac

SEQ ID NO: 6 - DNA, 15 bp, Artificial Sequence
  PNA probe Let-7f
acaatctactacctc

SEQ ID NO: 7 - DNA, 15 bp, Artificial Sequence
  PNA probe Let-7g
tgtacaaactactac

SEQ ID NO: 8 - DNA, 15 bp, Artificial Sequence
  PNA probe MiR-16
atttacgtgctgcta

SEQ ID NO: 9 - DNA, 15 bp, Artificial Sequence
  PNA probe MiR-21
cagtctgataagcta

SEQ ID NO: 10 - DNA, 15 bp, Artificial Sequence
  PNA probe MiR-24
tgctgaactgagcca

SEQ ID NO: 11 - DNA, 15 bp, Artificial Sequence
  PNA probe MiR-222
tagccagatgtagct

SEQ ID NO: 12 - DNA, 15 bp, Artificial Sequence
  PNA probe MiR-125b
ttagggtctcaggga

SEQ ID NO: 13 - DNA, 15 bp, Artificial Sequence
  PNA probe MiR-143
cagtgcttcatctca

SEQ ID NO: 14 - DNA, 15 bp, Artificial Sequence
  PNA probe MiR-142-5p
gctttctactttatg

SEQ ID NO: 15 - DNA, 15 bp, Artificial Sequence
  PNA probe MiR-142-3p
gtaggaaacactaca

SEQ ID NO: 16 - DNA, 15 bp, Artificial Sequence
  PNA probe MiR-155
cacgattagcattaa

SEQ ID NO: 17 - DNA, 15 bp, Artificial Sequence
  PNA probe MiR-15a
cattatgtgctgcta

SEQ ID NO: 18 - DNA, 15 bp, Artificial Sequence
  PNA probe MiR-145
ctgggaaaactggac

SEQ ID NO: 19 - DNA, 15 bp, Artificial Sequence
  PNA probe MiR-196a-1
caacaacatgaaact

SEQ ID NO: 20 - DNA, 15 bp, Artificial Sequence
  PNA probe MiR-196a-2
acaacatgaaactac

SEQ ID NO: 21 - DNA, 15 bp, Artificial Sequence
  PNA probe MiR-196b-1
acaacaggaaactac

SEQ ID NO: 22 - DNA, 15 bp, Artificial Sequence
  PNA probe MiR-196b-2
acaacaggaatctac

SEQ ID NO: 23 - DNA, 15 bp, Artificial Sequence
  PNA probe MiR-196b-3
acaacaggatactac

SEQ ID NO: 24 - DNA, 16 bp, Artificial Sequence
  PNA probe MiR-19a-1
agttttgcatagattt

SEQ ID NO: 25 - DNA, 17 bp, Artificial Sequence
  PNA probe MiR-19a-2
gttttgcatagatttgc

SEQ ID NO: 26 - DNA, 15 bp, Artificial Sequence
  PNA probe MiR-19b-1
gttttgcatggattt

SEQ ID NO: 27 - DNA, 13 bp, Artificial Sequence
  PNA probe MiR-19b-2
tgcatggatttgc

SEQ ID NO: 28 - DNA, 13 bp, Artificial Sequence
  PNA probe MiR-221-1
cccagcagacaat

SEQ ID NO: 29 - DNA, 12 bp, Artificial Sequence
  PNA probe MiR-221-2
gacaatgtagct

SEQ ID NO: 30 - DNA, 12 bp, Artificial Sequence
  PNA probe MiR-221-3
ccagcagacaat

SEQ ID NO: 31 - DNA, 15 bp, Artificial Sequence
  PNA probe MiR-181a
actcaccgacagcgt

SEQ ID NO: 32 - DNA, 15 bp, Artificial Sequence
  PNA probe MiR-181b
cccaccgacagcaat

SEQ ID NO: 33 - DNA, 15 bp, Artificial Sequence
  PNA probe MiR-181c
actcaccgacaggtt

SEQ ID NO: 34 - DNA, 15 bp, Artificial Sequence
  PNA probe MiR-18
tctgcactagatgca

SEQ ID NO: 35 - DNA, 15 bp, Artificial Sequence
  PNA probe MiR-224
taaacggaaccacta

SEQ ID NO: 36 - DNA, 15 bp, Artificial Sequence
  PNA probe MiR-199b
gaacagatagtctaa

SEQ ID NO: 37 - DNA, 15 bp, Artificial Sequence
  PNA probe MiR-195
gccaatatttctgtg

SEQ ID NO: 38 - DNA, 15 bp, Artificial Sequence
  PNA probe MiR-200a
tccagcactgtccgg

SEQ ID NO: 39 - DNA, 15 bp, Artificial Sequence
  PNA probe MiR-146a
aacccatggaattca

SEQ ID NO: 40 - DNA, 15 bp, Artificial Sequence
  PNA probe MiR-372
tcaaatgtcgcagca

SEQ ID NO: 41 - DNA, 15 bp, Artificial Sequence
  PNA probe MiR-373
ccccaaaatcgaagc

SEQ ID NO: 42 - DNA, 15 bp, Artificial Sequence
  PNA probe MiR-20a
ctacctgcactataa

SEQ ID NO: 43 - DNA, 15 bp, Artificial Sequence
  PNA probe MiR-21
tcaacatcagtctga

SEQ ID NO: 44 - DNA, 15 bp, Artificial Sequence
  PNA probe MiR-22
acagttcttcaactg

SEQ ID NO: 45 - DNA, 14 bp, Artificial Sequence
  PNA probe MiR-189
tcagctcagtagca

SEQ ID NO: 46 - DNA, 15 bp, Artificial Sequence
  PNA probe MiR-29b
aacactgatttcaaa

SEQ ID NO: 47 - DNA, 15 bp, Artificial Sequence
  PNA probe MiR-let7i
acagcacaaactact

SEQ ID NO: 48 - DNA, 18 bp, Artificial Sequence
  PNA probe MiR-1
tacatacttctttacatt

SEQ ID NO: 49 - DNA, 13 bp, Artificial Sequence
  PNA probe MiR-100, n represents 3-nitropyrrole or 5-nitroindole
cacaagttcggnt

SEQ ID NO: 50 - DNA, 21 bp, Artificial Sequence
  PNA probe MiR-101
ttcagttatcacagtactgta

SEQ ID NO: 51 - DNA, 14 bp, Artificial Sequence
  PNA probe MiR-103, n represents 3-nitropyrrole or 5-nitroindole
tcatagccctgnta

SEQ ID NO: 52 - DNA, 15 bp, Artificial Sequence
  PNA probe MiR-106a
ctacctgcactgtaa

SEQ ID NO: 53 - DNA, 17 bp, Artificial Sequence
  PNA probe MiR-106b
atctgcactgtcagcac

SEQ ID NO: 54 - DNA, 13 bp, Artificial Sequence
  PNA probe MiR-107, n represents 3-nitropyrrole or 5-nitroindole
ntgatagccctgt

SEQ ID NO: 55 - DNA, 14 bp, Artificial Sequence
  PNA probe MiR-10a
attcggatctacag

SEQ ID NO: 56 - DNA, 13 bp, Artificial Sequence
  PNA probe MiR-10b, n represents 3-nitropyrrole or 5-nitroindole
tncggttctacag

SEQ ID NO: 57 - DNA, 18 bp, Artificial Sequence
  PNA probe MiR-122
caaacaccattgtcacac

SEQ ID NO: 58 - DNA, 15 bp, Artificial Sequence
  PNA probe MiR-124a
tggcattcaccgcgt

SEQ ID NO: 59 - DNA, 14 bp, Artificial Sequence
  PNA probe MiR-125a
tcacaggttaaagt

SEQ ID NO: 60 - DNA, 21 bp, Artificial Sequence
  PNA probe MiR-126
gcattattactcacggtacga

SEQ ID NO: 61 - DNA, 15 bp, Artificial Sequence
  PNA probe MiR-127-3p
agccaagctcagacg

SEQ ID NO: 62 - DNA, 16 bp, Artificial Sequence
  PNA probe MiR-127-5p
agccctctgagcttca

SEQ ID NO: 63 - DNA, 14 bp, Artificial Sequence
  PNA probe MiR-128
ccggttcactgtga

SEQ ID NO: 64 - DNA, 18 bp, Artificial Sequence
  PNA probe MiR-132
cgatcatggctgtagact

SEQ ID NO: 65 - DNA, 16 bp, Artificial Sequence
  PNA probe MiR-133a
ctggttgaagtggacc

SEQ ID NO: 66 - DNA, 15 bp, Artificial Sequence
  PNA probe MiR-133b
tagctggttgaagtg

SEQ ID NO: 67 - DNA, 15 bp, Artificial Sequence
  PNA probe MiR-134
cccctctggtcaacc

SEQ ID NO: 68 - DNA, 16 bp, Artificial Sequence
  PNA probe MiR-135a
tcacataggaataaaa

SEQ ID NO: 69 - DNA, 15 bp, Artificial Sequence
  PNA probe MiR-135b
tcacataggaatgaa

SEQ ID NO: 70 - DNA, 18 bp, Artificial Sequence
  PNA probe MiR-136
ccatcatcaaaacaaatg

SEQ ID NO: 71 - DNA, 20 bp, Artificial Sequence
  PNA probe MiR-137
cgcgtattcttaatcaataa

SEQ ID NO: 72 - DNA, 17 bp, Artificial Sequence
  PNA probe MiR-140-3p
cgtggttctaccctgtg

SEQ ID NO: 73 - DNA, 18 bp, Artificial Sequence
  PNA probe MiR-140-5p
ccatagggtaaaaccact

SEQ ID NO: 74 - DNA, 15 bp, Artificial Sequence
  PNA probe MiR-141
ccatctttaccagac

SEQ ID NO: 75 - DNA, 15 bp, Artificial Sequence
  PNA probe MiR-146b
agcctatggaattca

SEQ ID NO: 76 - DNA, 14 bp, Artificial Sequence
  PNA probe MiR-148a
agttctgtagtgca

SEQ ID NO: 77 - DNA, 10 bp, Artificial Sequence
  PNA probe MiR-149
acacggagcc

SEQ ID NO: 78 - DNA, 18 bp, Artificial Sequence
  PNA probe MiR-150
cactggtacaatggttgg

SEQ ID NO: 79 - DNA, 17 bp, Artificial Sequence
  PNA probe MiR-151
aggagcttcagtctagt

SEQ ID NO: 80 - DNA, 18 bp, Artificial Sequence
  PNA probe MiR-153
tcacttttgtgactatgc

SEQ ID NO: 81 - DNA, 17 bp, Artificial Sequence
  PNA probe MiR-154
ggcaacacggataacct

SEQ ID NO: 82 - DNA, 17 bp, Artificial Sequence
  PNA probe MiR-15b
tgtaaaccatgatgtgc

SEQ ID NO: 83 - DNA, 16 bp, Artificial Sequence
  PNA probe MiR-17-3p
cactgtaagcactttg

SEQ ID NO: 84 - DNA, 18 bp, Artificial Sequence
  PNA probe MiR-17-5p
acaagtgccttcactgca

SEQ ID NO: 85 - DNA, 15 bp, Artificial Sequence
  PNA probe MiR-181d, n represents 3-nitropyrrole or 5-nitroindole
ccaccgacaacanat

SEQ ID NO: 86 - DNA, 15 bp, Artificial Sequence
  PNA probe MiR-182
tgtgagttctaccat

SEQ ID NO: 87 - DNA, 13 bp, Artificial Sequence
  PNA probe MiR-183
taccagtgccata

SEQ ID NO: 88 - DNA, 18 bp, Artificial Sequence
  PNA probe MiR-185
gaactgcctttctctcca

SEQ ID NO: 89 - DNA, 15 bp, Artificial Sequence
  PNA probe MiR-186
aagcccaaaaggaga

SEQ ID NO: 90 - DNA, 16 bp, Artificial Sequence
  PNA probe MiR-188-3p
caaaccctgcatgtgg

SEQ ID NO: 91 - DNA, 13 bp, Artificial Sequence
  PNA probe MiR-188-5p
tccaccatgcaag

SEQ ID NO: 92 - DNA, 18 bp, Artificial Sequence
  PNA probe MiR-18b
ctgcactagatgcacctt

SEQ ID NO: 93 - DNA, 18 bp, Artificial Sequence
  PNA probe MiR-190b
aacccaatatcaaacata

SEQ ID NO: 94 - DNA, 15 bp, Artificial Sequence
  PNA probe MiR-191, n represents 3-nitropyrrole or 5-nitroindole
tntgggattccgttg

SEQ ID NO: 95 - DNA, 15 bp, Artificial Sequence
  PNA probe MiR-192
ggctgtcaattcata

SEQ ID NO: 96 - DNA, 18 bp, Artificial Sequence
  PNA probe MiR-194
acatggagttgctgttac

SEQ ID NO: 97 - DNA, 15 bp, Artificial Sequence
  PNA probe MiR-197, n represents 3-nitropyrrole or 5-nitroindole
ctgggtggnganggt

SEQ ID NO: 98 - DNA, 17 bp, Artificial Sequence
  PNA probe MiR-198
tatctcccctctggacc

SEQ ID NO: 99 - DNA, 14 bp, Artificial Sequence
  PNA probe MiR-199a
caggtagtctgaac

SEQ ID NO: 100 - DNA, 17 bp, Artificial Sequence
  PNA probe MiR-199a-3p
taaccaatgtgcagact

SEQ ID NO: 101 - DNA, 12 bp, Artificial Sequence
  PNA probe MiR-200b
tcattaccaggc

SEQ ID NO: 102 - DNA, 15 bp, Artificial Sequence
  PNA probe MiR-200c
atcattacccgtcag

SEQ ID NO: 103 - DNA, 18 bp, Artificial Sequence
  PNA probe MiR-202
tcccatgccctatacctc

SEQ ID NO: 104 - DNA, 18 bp, Artificial Sequence
  PNA probe MiR-203
ctagtggtcctaaacatt

SEQ ID NO: 105 - DNA, 16 bp, Artificial Sequence
  PNA probe MiR-204
aggcataggattacaa

SEQ ID NO: 106 - DNA, 16 bp, Artificial Sequence
  PNA probe MiR-205
cagactccgttggaat

SEQ ID NO: 107 - DNA, 17 bp, Artificial Sequence
  PNA probe MiR-206
cacttccttacattcca

SEQ ID NO: 108 - DNA, 15 bp, Artificial Sequence
  PNA probe MiR-210
ttagccgctgtcaca

SEQ ID NO: 109 - DNA, 16 bp, Artificial Sequence
  PNA probe MiR-214
ctgcctgtctgtgcct

SEQ ID NO: 110 - DNA, 20 bp, Artificial Sequence
  PNA probe MiR-215
gtctgtcaattcataggtca

SEQ ID NO: 111 - DNA, 15 bp, Artificial Sequence
  PNA probe MiR-216a
tcacagttgccagct

SEQ ID NO: 112 - DNA, 18 bp, Artificial Sequence
  PNA probe MiR-216b
tcacatttgcctgcagag

SEQ ID NO: 113 - DNA, 19 bp, Artificial Sequence
  PNA probe MiR-218
acatggttagatcaagcac

SEQ ID NO: 114 - DNA, 17 bp, Artificial Sequence
  PNA probe MiR-219
ttgcgtttggacaatca

SEQ ID NO: 115 - DNA, 13 bp, Artificial Sequence
  PNA probe MiR-223
ttgacaaactgac

SEQ ID NO: 116 - DNA, 16 bp, Artificial Sequence
  PNA probe MiR-23a
ttttttggaaatccct

SEQ ID NO: 117 - DNA, 16 bp, Artificial Sequence
  PNA probe MiR-25
tcagaccgagacaagt

SEQ ID NO: 118 - DNA, 15 bp, Artificial Sequence
  PNA probe MiR-26a
gcctatcctggatta

SEQ ID NO: 119 - DNA, 16 bp, Artificial Sequence
  PNA probe MiR-26b
tatcctgaattactta

SEQ ID NO: 120 - DNA, 14 bp, Artificial Sequence
  PNA probe MiR-27a
gcggaacttagcca

SEQ ID NO: 121 - DNA, 12 bp, Artificial Sequence
  PNA probe MiR-27b
gcagaacttagc

SEQ ID NO: 122 - DNA, 15 bp, Artificial Sequence
  PNA probe MiR-28-5p
ctcaatagactgtga

SEQ ID NO: 123 - DNA, 16 bp, Artificial Sequence
  PNA probe MiR-296-3p
cctccacccaaccctc

SEQ ID NO: 124 - DNA, 15 bp, Artificial Sequence
  PNA probe MiR-296-5p
ttgagggttggccct

SEQ ID NO: 125 - DNA, 15 bp, Artificial Sequence
  PNA probe MiR-29a
taaccgatttcagat

SEQ ID NO: 126 - DNA, 15 bp, Artificial Sequence
  PNA probe MiR-29c
accgatttcaaatgg

SEQ ID NO: 127 - DNA, 15 bp, Artificial Sequence
  PNA probe MiR-30a
cttccagtcgaggat

SEQ ID NO: 128 - DNA, 17 bp, Artificial Sequence
  PNA probe MiR-30b, n represents 3-nitropyrrole or 5-nitroindole
agctgagtgtagnntgt

SEQ ID NO: 129 - DNA, 12 bp, Artificial Sequence
  PNA probe MiR-30c
gctgagagtgta

SEQ ID NO: 130 - DNA, 18 bp, Artificial Sequence
  PNA probe MiR-31
cagctatgccagcatctt

SEQ ID NO: 131 - DNA, 16 bp, Artificial Sequence
  PNA probe MiR-342-3p
ggtgcgatttctgtgt

SEQ ID NO: 132 - DNA, 17 bp, Artificial Sequence
  PNA probe MiR-342-5p
caatcacagatagcacc

SEQ ID NO: 133 - DNA, 17 bp, Artificial Sequence
  PNA probe MiR-34a
acaaccagctaagacac

SEQ ID NO: 134 - DNA, 21 bp, Artificial Sequence
  PNA probe MiR-368
acgtggaattacctctatgtt

SEQ ID NO: 135 - DNA, 15 bp, Artificial Sequence
  PNA probe MiR-375
tcacgcgagcctaac

SEQ ID NO: 136 - DNA, 21 bp, Artificial Sequence
  PNA probe MiR-488
gaccaataaatagcctttcaa

SEQ ID NO: 137 - DNA, 17 bp, Artificial Sequence
  PNA probe MiR-7
aaatcactagtcttcca

SEQ ID NO: 138 - DNA, 18 bp, Artificial Sequence
  PNA probe MiR-9
catacagctagataacca

SEQ ID NO: 139 - DNA, 16 bp, Artificial Sequence
  PNA probe MiR-9*
ttcggttatctagctt

SEQ ID NO: 140 - DNA, 13 bp, Artificial Sequence
  PNA probe MiR-92a, n represents 3-nitropyrrole or 5-nitroindole
ccnggacaagtgc

SEQ ID NO: 141 - DNA, 14 bp, Artificial Sequence
  PNA probe MiR-92b, n represents 3-nitropyrrole or 5-nitroindole
ccggnacgagtgcn

SEQ ID NO: 142 - DNA, 15 bp, Artificial Sequence
  PNA probe MiR-93
tgcacgaacagcact

SEQ ID NO: 143 - DNA, 18 bp, Artificial Sequence
  PNA probe MiR-95
tgctcaataaatacccgt

SEQ ID NO: 144 - DNA, 15 bp, Artificial Sequence
  PNA probe MiR-99a
cacaagatcggattt

(19) United States
(12) Patent Application Publication
(10) Pub. No.: US 2011/0111416 A1
(43) Pub. Date: May 12, 2011

(54) Peptide Nucleic Acid Probes, Kits and Methods for Expression Profiling of Micrornas

(75) Inventors: Hee Kyung Park, Daejeon (KR); Jae Jin Choi, Daejeon (KR)

(73) Assignee: PANAGENE INC., Gyeongsangbuk-do (KR)

(21) Appl. No.: 12/991,364

(22) PCT Filed: Mar. 27, 2009
(86) PCT No.: PCT/KR09/01569

§ 371 Date: Nov. 5, 2010

(30) Foreign Application Priority Data
May 6, 2008 (KR) 10 2008 0042003

(51) Int. Cl. C12Q 1/68; C07K 9/00

(52) U.S. Cl. 435/6; 530/322

(57) ABSTRACT

Disclosed are peptide nucleic acid (PNA) probes, a kit and a method for expression profiling of microRNAs (miRNAs), which play an important role in regulation of expression of genes encoding proteins.

### TECHNICAL FIELD

The present invention relates to expression profiling of microRNAs (miRNAs), and more particularly, to peptide nucleic acid (PNA) probes for expression profiling of miRNAs, which play an important role in regulation of expression of genes encoding proteins, a kit therefor including the same, and a method therefor using the same.

### BACKGROUND ART

MicroRNAs (miRNAs) are single-stranded RNA molecules of 21-25 nucleotides, which regulate gene expression of eukaryotes. They bind to 3′ untranslated region (UTR) of mRNA for a specific gene and regulate its translational process. MiRNA has been received a great attention since some genes were found to control developmental stages in Caenorhabditis elegans in 1993. Among them, let-7 and lin-4 were identified as non-coding RNAs that do not produce proteins. These RNAs were termed as small temporal RNAs (stRNA) because they are expressed in a specific developmental stage and control the development. The miRNAs play a critical role in temporally regulating cellular development by inducing switching-off of target molecules. Until recently, hundreds of miRNAs were identified. They are thought to be involved in the regulation of cell growth, differentiation and death in worms, flies and humans. More than 500 miRNAs were identified in the human genome only (see the literature [Griffiths-Jones et al., 2008, Nucleic Acids Research, 36(Database issue):D154-158]).

Biosynthesis of miRNA is initiated by transcription by RNA polymerase II. The process proceeds in two stages. First, a primary miRNA transcript (pri-miRNA) is processed into a pre-miRNA of stem & loop structure, which has the length of about 70-90 nucleotides, in the nucleus by an RNase III type enzyme called Drosha. Then, the pre-miRNA is transferred to the cytosol and cleaved with an enzyme called Dicer to form a mature miRNA of 21-25 nucleotides. Since some miRNAs have highly interspecifically conserved base sequences, they are thought to be involved in important biological activities, and so extensive studies are performed thereon. Recently, many researches showed that miRNA plays important roles not only in cancer cells and stem cells but also in regulation of cell proliferation, differentiation and death, as well as regulation of lipid metabolism. However, many functions of miRNA are still unknown, and a lot of researches are actively ongoing thereon. MiRNA is one of thousands of small RNA fragments existing in cells.

Through expression profiling of miRNAs, it is possible to screen miRNAs closely associated with specific diseases or cancers, and thus screened miRNAs can be utilized as biomarkers to diagnose and prognose the diseases (see the literature [Bartels et al., 2009, MicroRNAs: Novel Biomarkers for Human Cancer. Clin Chem. 55(4):[Epub ahead of print]], [Nelson et al. 2008, MicroRNAs and cancer: past, present, and potential future. Mol Cancer Ther. 7(12):3655-60], [Sassen et al. 2008, MicroRNA: implications for cancer. Virchows Arch. 452(1):1-10], [Gilad et al. 2008, Serum MicroRNAs Are Promising Novel Biomarkers. PLoS ONE. 3(9): e3148], [Wu et al. 2007, MicroRNA and cancer: Current status and prospective. Int J Cancer. 120(5):953-60], and [Stenvang et al., 2008, The utility of LNA in microRNA-based cancer diagnostics and therapeutics. Seminars in Cancer Biology. 18:89-102]).

Therefore, expression profiling of miRNA is of great importance.

Certain miRNAs are related to specific cancers (see the literature [Yang et al. 2008, MicroRNA Microarray Identifies Let-7i as a Novel Biomarker and Therapeutic Target in Human Epithelial Ovarian Cancer. Cancer Res. 68(24):10307-14], [Yan et al. 2008, MicroRNA miR-21 overexpression in human breast cancer is associated with advanced clinical stage, lymph node metastasis and patient poor prognosis. RNA. 14(10:2348-60], [Bloomston et al. 2008, MicroRNA expression patterns to differentiate pancreatic adenocarcinoma from normal pancreas and chronic pancreatitis. J. Am. Med. Assoc. 297(17):1901-8], [Akao et al. 2007, MicroRNA-143 and -145 in colon cancer. DNA Cell Biol. 26(5):311-20], [Yanaihara et al. 2006, Unique microRNA molecular profiles in lung cancer diagnosis and prognosis. Cancer Cell. 9(3):189-98], [Pekarsky et al. 2006, Tcl1 expression in chronic lymphocytic leukemia is regulated by miR-29 and miR-181. Cancer Res. 66(24):11590-3], [Iorio et al. 2007, MicroRNA signatures in human ovarian cancer. Cancer Res. 67(18):8699-707], [Laios et al. 2008, Potential role of miR-9 and miR-223 in recurrent ovarian cancer. Mol Cancer. 7:35], and [Roldo et al. 2006, MicroRNA expression abnormalities in pancreatic endocrine and acinar tumors are associated with distinctive pathologic features and clinical behavior. J Clin Oncol. 24(29):4677-84]).

At present, a lot of experimental techniques are developed for expression profiling of miRNAs. Commonly, northern blot, real-time polymerase chain reaction (PCR), etc. are used for expression profiling (see the literature [Boutla et al., 2003, Developmental defects by antisense-mediated inactivation of micro-RNAs 2 and 13 in Drosophila and the identification of putative target genes. Nucleic Acids Research, 31(17): 4973-4980]). Although the northern blot is a fundamental and essential method for studying expression of miRNAs, it requires a large amount of RNA to detect small RNA fragments with probes, is time-, labor- and skill-intensive and not cost-effective, and has limitation in that only a single miRNA expression pattern can be detected at a time (see the literature [Kloosterman W P et al., 2006, Devel. Cell, 11(4):441-50] and [Kloosterman W P et al., 2006, Nat. Methods, 3(1):27-29]). A variety of DNA microarray chips for multiplex expression profiling of various genes at a time are developed and utilized. The DNA chip has densely immobilized DNA probes designed based on known genetic information on the surface of a solid support, and their hybridization with target nucleic acids to be analyzed on the chip is detected from fluorescence. The miRNA microarray enables simultaneous analysis of miRNAs expressed specifically from various cells or tissues. Using a DNA chip, a variety of genetic information can be analyzed through just one experiment.

Therefore, this technique is very useful in diagnosis of diseases (see the literatures [Kim K et al., 2006, Gynecologic Oncology, 100:38-43] and [BeuVink et al., 2007, Nucleic Acids Research, vol 35, No 7]). The DNA chip is the most effective analytic and diagnostic tool developed hitherto, but it still has the following drawbacks.

First, since DNA probes are unstable biologically (against nucleases) and chemically (against acids or bases), the DNA chip has a low stability.

Second, it is difficult to detect variation in a single nucleotide, such as single-nucleotide polymorphism (SNP) or point mutation.

Third, for detection of widely scattered variations or expression profiling of whole genes, fragmentation of target nucleic acids and complicated labeling process, including labeling of each fragmented target nucleic acids with fluorescent dyes or amplification of target nucleic acids with addition of fluorescent dyes, are required.

Peptide nucleic acid (PNA) is a DNA analogue whose nucleobases are linked by peptide bonds, not by phosphate bonds, and was first reported in 1991 (see the literature [Nielsen P E et al., 1991, Science, 254:1497-1500]).

PNA is not naturally occurring but synthesized artificially through a chemical process. PNA is hybridized with a natural nucleic acid with a complementary base sequence to form a double strand. Given the same length, a PNA/DNA double strand is more stable than a DNA/DNA double strand, and a PNA/RNA double strand is more stable than a DNA/RNA double strand. Further, PNA has a higher detectability for point mutation or SNP, because its double strand is unstablized at a larger extent from a single nucleotide mismatch, than natural nucleic acids. The peptide backbone is often composed of repeating N-(2-aminoethyl)glycine units linked by amide bonds. Such PNA has the electrically neutral backbone, differently from negatively charged natural nucleic acids. PNAs with other backbones than the N-(2-aminoethyl)glycine repeating units are also known (see the literature [P. E. Nielsen and M. Egholm “An Introduction to PNA” in P. E. Nielsen (Ed.) “Peptide Nucleic Acids: Protocols and Applications” 2nd Ed. Page 9 (Horizon Bioscience, 2004)]). The four nucleobases of PNA occupy similar space as those of DNA, and the distance between the nucleobases is almost identical to that in natural nucleic acids. PNAs are more chemically stable than natural nucleic acids. In addition, they are more biologically stable because they are not degraded by nucleases or proteases. Since PNA is electrically neutral, the stability of the PNA/DNA and PNA/RNA double strands is not affected by the salt concentration. In addition, PNA has many advantages in that it can be readily labeled with a fluorescent dye, if necessary, and have an increased solubility by binding with ions. With these advantages, PNA could be used widely in the field of cancer cell research, pathogenic microbiology, virology, or the like, as a means for detecting mutations that cause genetic disorders or for early diagnosis of infection with pathogenic bacteria or viruses. As described above, PNA, which has a high hybridization ability and stability while retaining the functions of DNA or RNA, is recognized as a promising alternative to DNA that can complement the drawbacks of DNA. Thus, extensive researches are ongoing on its applications for assays, diagnoses, and the like (see Brandt O et al., 2004, Trends in Biotechnology. 22:617-622;Raymond F et al., 2005, BMC Biotechnology, 5:10).

### DISCLOSURE OF INVENTION

### Technical Problem

The inventors have designed peptide nucleic acid (PNA) probes capable of specifically binding to their target microRNAs (miRNAs) and enabling expression profiling thereof, and manufactured a PNA chip. They have confirmed that, using them, expression profiling of miRNAs could be achieved with high specificity and sensitivity, and thus, completed the present invention.

Accordingly, an object of the present invention is to provide PNA probes capable of profiling expression of miRNAs with high specificity and sensitivity.

Another object of the present invention is to provide a kit for expression profiling of miRNAs, including the PNA probes.

Still another object of the present invention is to provide a method for expression profiling of miRNAs using the PNA probes.

### Technical Solution

To achieve the objects of the present invention, the present invention provides peptide nucleic acid (PNA) probes capable of specifically binding to target microRNAs (miRNAs), each of which consists of 13 to 22 bases and includes base sequences complementary to 3 to 10 base sequences in 5′ seed of its target miRNA. The PNA probe according to the present invention can specifically bind to a miRNA selected from the group consisting of let-7a, let-7b, let-7c, let-7d, let-7e, let-7f, let-7g, miR-1, miR-1b, miR-1d, miR-2, miR-7, miR-7b, miR-9, miR-10b, miR-12a, miR-15a, miR-15b, miR-16, miR-16-1, miR-17-5p, miR-17-92 cluster, miR-18, miR-19a, miR-19b, miR-20a, miR-21, miR-22, miR-23a, miR-23b, miR-24, miR-29, miR-29b, miR-29c, miR-31, miR-34, miR-34a, miR-102, miR-103, miR-107, miR-122, miR-124, miR-124b, miR-125a, miR-125b, miR-127, miR-128, miR-133b, miR-135b, miR-142-5p, miR-142-3p, miR-143, miR-145, miR-146a, miR-151, miR-153, miR-155, miR-181, miR-181a, miR-181b, miR-181c, miR-182, miR-183, miR-184, miR-186, miR-189, miR-195, miR-196a, miR-196b, miR-199b, miR-200a, miR-200b, miR-200c, miR-206, miR-208, miR-211, miR-212, miR-213, miR-214, miR-215, miR-221, miR-222, miR-223, miR-224, miR-296, miR-301, miR-363, miR-372, miR-373, miR-376, miR-380 and miR-430.

Particularly, it may consist of any one of the nucleotide sequences as set forth in SEQ ID Nos. 1 to 144.

The present invention also provides a kit for expression profiling of miRNAs, which includes one or more of the PNA probe(s).

The present invention further provides a method for expression profiling of miRNAs, comprising:

(1) introducing a reaction sample containing miRNAs to the kit including one or more of the PNA probe(s);

(2) performing hybridization reaction between the PNA probe(s) and the target miRNA(s); and

(3) detecting a signal from the hybridization.

### Advantageous Effects

According to the present invention, expression profiling of microRNAs (miRNAs), which are involved in the regulation of expression of important genes, can be rapidly performed with high sensitivity and specificity.

Further, since the peptide nucleic acid (PNA) itself, which is used as a probe, is extremely stable against biological enzymes and physical factors, it is not influenced by environmental changes or other factors. Thus, it is expected to successfully replace DNA probes in commercial expression profiling of miRNAs.

### BRIEF DESCRIPTION OF DRAWINGS

The above and other objects, features and advantages of the present invention will become apparent from the following description of preferred embodiments given in conjunction with the accompanying drawings, in which:

FIG. 1 is a schematic diagram showing the kinds and positions of probes on the peptide nucleic acid (PNA) chip in accordance with an embodiment of the present invention.

FIG. 2 is a graph showing the results of hybridization of microRNA (miRNA) let-7 family with a single different base from one another, as targets, on the chip in accordance with an embodiment of the present invention.

FIG. 3 is a graph and an image showing the results of hybridization of miRNA 16, as a target, on the chip in accordance with an embodiment of the present invention.

FIG. 4 is a graph and an image showing the results of hybridization of miRNA 21, as a target, on the chip in accordance with an embodiment of the present invention.

FIG. 5 is a graph and an image showing the results of hybridization of miRNA 143, as a target, on the chip in accordance with an embodiment of the present invention.

FIG. 6 is a graph and an image showing the results of hybridization of miRNA 142-3p, as a target, on the chip in accordance with an embodiment of the present invention.

FIG. 7 is a graph showing the results of hybridization of synthetic miRNA 222 diluted to various concentrations.

### BEST MODE FOR CARRYING OUT THE INVENTION

Hereinafter, the embodiments of the present invention will be described in detail.

The peptide nucleic acid (PNA) probes for expression profiling of microRNAs (miRNAs), and the kit and the method for expression profiling thereof have been completed according to the following procedures.

1. Obtainment of miRNA Sequences

MiRNA sequences associated with the regulation of various diseases and cancers were obtained from databases including http://microna.sanger.ac.uk/sequences/, http://genome.ucsc.edu/, http://www.bioinfo.rpi.edu/zukerm/rna/mfold-3.html, http://www.ncbi.nim.nih.gov/, and the like. Examples of the target miRNA to be detected in accordance with the present invention include let-7a, let-7b, let-7c, let-7d, let-7e, let-7f, let-7g, miR-16, miR-21, miR-24, miR-222, miR-125b, miR-143, miR-142-5p, miR-142-3p, miR-155, miR-15a, miR-145, miR-196a, miR-196b, miR-19a, miR-19b, miR-221, miR-181a, miR-181b, miR-181c, miR-18, miR-224, miR-199b, miR-195, miR-200a, miR-146a, miR-372, miR-373, miR-20a, miR-21, miR-22, miR-189 and miR-29b.

2. Design and Manufacture of PNA Probes

PNA probes that could complementarily bind to miRNAs were designed for expression profiling of miRNAs. Table 1 shows SEQ ID Nos., names and base sequences of the PNA probes according to the present invention.

TABLE 1

| SEQ ID No. | Probe Name | Sequence (N to C) |
|---|---|---|
| 1 | let-7a | tatacaacctactac |
| 2 | let-7b | ccacacaacctacta |
| 3 | let-7c | catacaacctactac |
| 4 | let-7d | tatgcaacctactac |
| 5 | let-7e | tatacaacctcctac |
| 6 | let-7f | acaatctactacctc |
| 7 | let-7g | tgtacaaactactac |
| 8 | miR-16 | atttacgtgctgcta |
| 9 | miR-21 | cagtctgataagcta |
| 10 | miR-24 | tgctgaactgagcca |
| 11 | miR-222 | tagccagatgtagct |
| 12 | miR-125b | ttagggtctcaggga |
| 13 | miR-143 | cagtgcttcatctca |
| 14 | miR-142-5p | gctttctactttatg |
| 15 | miR-142-3p | gtaggaaacactaca |
| 16 | miR-155 | cacgattagcattaa |
| 17 | miR-15a | cattatgtgctgcta |
| 18 | miR-145 | ctgggaaaactggac |
| 19 | miR-196a-1 | caacaacatgaaact |
| 20 | miR-196a-2 | acaacatgaaactac |
| 21 | miR-196b-1 | acaacaggaaactac |
| 22 | miR-196b-2 | acaacaggaatctac |
| 23 | miR-196b-3 | acaacaggatactac |
| 24 | miR-19a-1 | agttttgcatagattt |
| 25 | miR-19a-2 | gttttgcatagatttgc |
| 26 | miR-19b-1 | gttttgcatggattt |
| 27 | miR-19b-2 | tgcatggatttgc |
| 28 | miR-221-1 | cccagcagacaat |
| 29 | miR-221-2 | gacaatgtagct |
| 30 | miR-221-3 | ccagcagacaat |
| 31 | miR-181a | actcaccgacagcgt |
| 32 | miR-181b | cccaccgacagcaat |
| 33 | miR-181c | actcaccgacaggtt |
| 34 | miR-18 | tctgcactagatgca |
| 35 | miR-224 | taaacggaaccacta |
| 36 | miR-199b | gaacagatagtctaa |
| 37 | miR-195 | gccaatatttctgtg |
| 38 | miR-200a | tccagcactgtccgg |
| 39 | miR-146a | aacccatggaattca |
| 40 | miR-372 | tcaaatgtcgcagca |
| 41 | miR-373 | ccccaaaatcgaagc |
| 42 | miR-20a | ctacctgcactataa |
| 43 | miR-21 | tcaacatcagtctga |
| 44 | miR-22 | acagttcttcaactg |
| 45 | miR-189 | tcagctcagtagca |
| 46 | miR-29b | aacactgatttcaaa |
| 47 | MiR-let7i | acagcacaaactact |
| 48 | MiR-1 | tacatacttctttacatt |
| 49 | MiR-100 | cacaagttcggxt |
| 50 | MiR-101 | ttcagttatcacagtactgta |
| 51 | MiR-103 | tcatagccctgxta |
| 52 | MiR-106a | ctacctgcactgtaa |
| 53 | MiR-106b | atctgcactgtcagcac |
| 54 | MiR-107 | xtgatagccctgt |
| 55 | MiR-10a | attcggatctacag |
| 56 | MiR-10b | txcggttctacag |
| 57 | MiR-122 | caaacaccattgtcacac |
| 58 | MiR-124a | tggcattcaccgcgt |
| 59 | MiR-125a | tcacaggttaaagt |
| 60 | MiR-126 | gcattattactcacggtacga |
| 61 | MiR-127-3p | agccaagctcagacg |
| 62 | MiR-127-5p | agccctctgagcttca |
| 63 | MiR-128 | ccggttcactgtga |
| 64 | MiR-132 | cgatcatggctgtagact |
| 65 | MiR-133a | ctggttgaagtggacc |
| 66 | MiR-133b | tagctggttgaagtg |
| 67 | MiR-134 | cccctctggtcaacc |
| 68 | MiR-135a | tcacataggaataaaa |
| 69 | MiR-135b | tcacataggaatgaa |
| 70 | MiR-136 | ccatcatcaaaacaaatg |
| 71 | MiR-137 | cgcgtattcttaatcaataa |
| 72 | MiR-140-3p | cgtggttctaccctgtg |
| 73 | MiR-140-5p | ccatagggtaaaaccact |
| 74 | MiR-141 | ccatctttaccagac |
| 75 | MiR-146b | agcctatggaattca |
| 76 | MiR-148a | agttctgtagtgca |
| 77 | MiR-149 | acacggagcc |
| 78 | MiR-150 | cactggtacaatggttgg |
| 79 | MiR-151 | aggagcttcagtctagt |
| 80 | MiR-153 | tcacttttgtgactatgc |
| 81 | MiR-154 | ggcaacacggataacct |
| 82 | MiR-15b | tgtaaaccatgatgtgc |
| 83 | MiR-17-3p | cactgtaagcactttg |
| 84 | MiR-17-5p | acaagtgccttcactgca |
| 85 | MiR-181d | ccaccgacaacaxat |
| 86 | MiR-182 | tgtgagttctaccat |
| 87 | MiR-183 | taccagtgccata |
| 88 | MiR-185 | gaactgcctttctctcca |
| 89 | MiR-186 | aagcccaaaaggaga |
| 90 | MiR-188-3p | caaaccctgcatgtgg |
| 91 | MiR-188-5p | tccaccatgcaag |
| 92 | MiR-18b | ctgcactagatgcacctt |
| 93 | MiR-190b | aacccaatatcaaacata |
| 94 | MiR-191 | txtgggattccgttg |
| 95 | MiR-192 | ggctgtcaattcata |
| 96 | MiR-194 | acatggagttgctgttac |
| 97 | MiR-197 | ctgggtggxgaxggt |
| 98 | MiR-198 | tatctcccctctggacc |
| 99 | MiR-199a | caggtagtctgaac |
| 100 | MiR-199a-3p | taaccaatgtgcagact |
| 101 | MiR-200b | tcattaccaggc |
| 102 | MiR-200c | atcattacccgtcag |
| 103 | MiR-202 | tcccatgccctatacctc |
| 104 | MiR-203 | ctagtggtcctaaacatt |
| 105 | MiR-204 | aggcataggattacaa |
| 106 | MiR-205 | cagactccgttggaat |
| 107 | MiR-206 | cacttccttacattcca |
| 108 | MiR-210 | ttagccgctgtcaca |
| 109 | MiR-214 | ctgcctgtctgtgcct |
| 110 | MiR-215 | gtctgtcaattcataggtca |
| 111 | MiR-216a | tcacagttgccagct |
| 112 | MiR-216b | tcacatttgcctgcagag |
| 113 | MiR-218 | acatggttagatcaagcac |
| 114 | MiR-219 | ttgcgtttggacaatca |
| 115 | MiR-223 | ttgacaaactgac |
| 116 | MiR-23a | ttttttggaaatccct |
| 117 | MiR-25 | tcagaccgagacaagt |
| 118 | MiR-26a | gcctatcctggatta |
| 119 | MiR-26b | tatcctgaattactta |
| 120 | MiR-27a | gcggaacttagcca |
| 121 | MiR-27b | gcagaacttagc |
| 122 | MiR-28-5p | ctcaatagactgtga |
| 123 | MiR-296-3p | cctccacccaaccctc |
| 124 | MiR-296-5p | ttgagggttggccct |
| 125 | MiR-29a | taaccgatttcagat |
| 126 | MiR-29c | accgatttcaaatgg |
| 127 | MiR-30a | cttccagtcgaggat |
| 128 | MiR-30b | agctgagtgtagxxtgt |
| 129 | MiR-30c | gctgagagtgta |
| 130 | MiR-31 | cagctatgccagcatctt |
| 131 | MiR-342-3p | ggtgcgatttctgtgt |
| 132 | M1R-342-5p | caatcacagatagcacc |
| 133 | MiR-34a | acaaccagctaagacac |
| 134 | MiR-368 | acgtggaattacctctatgtt |
| 135 | MiR-375 | tcacgcgagcctaac |
| 136 | MiR-488 | gaccaataaatagcctttcaa |
| 137 | MiR-7 | aaatcactagtcttcca |
| 138 | MiR-9 | catacagctagataacca |
| 139 | MiR-9* | ttcggttatctagctt |
| 140 | MiR-92a | ccxggacaagtgc |
| 141 | MiR-92b | ccggxacgagtgcx |
| 142 | MiR-93 | tgcacgaacagcact |
| 143 | MiR-95 | tgctcaataaatacccgt |
| 144 | MiR-99a | cacaagatcggattt |

(x: 3-nitropyrrole or 5-nitroindole)

As can be seen from Table 1, the PNA probes according to the present invention consist of the nucleotide sequences as set forth in SEQ ID Nos. 1 to 144, complementary to their target miRNA sequences. The PNA probes according to the present invention consist of 13-22 bases and include 3-10, particularly 3-8, more particularly 8, base sequences complementary to 5′ seed of their target miRNAs, which is important in recognizing the target miRNAs. The PNA probes of SEQ ID Nos. 1 to 7 are designed to complementarily bind to miRNAs of let-7 family, including let-7a to let-7g having important regulatory functions in many tissues and including only a single different base from one another, and so are essentially included in the analysis of miRNAs. The probes were designed to investigate the specificity to accurately discriminate target miRNAs from ones with only one different base therefrom.

The PNA probes of SEQ ID Nos. 8 to 144 are designed to complementarily bind to the representative miRNAs closely associated with the regulation of cancers or important genes. The miRNAs used in embodiments of the present invention are only representatives, but the scope of the present invention is not limited thereby, and probes may be designed for various target miRNAs.

In a preferable embodiment, the PNA probe according to the present invention may have a multi-amine linker represented by the following Formula 1, capable of reacting with epoxy group, at N- and C-terminals, for efficient immobilization on a support(s), but the scope of the present invention is not limited thereby:

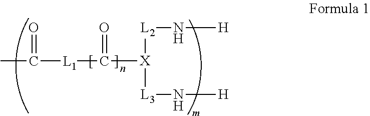

wherein

L₁, L₂ and L₃ independently of one another represent a chemical bond or a C₁-C₁₀ linear chain, wherein the C₁-C₁₀ linear chain may further include 1 to 3 oxygen(s);

X represents CH or N;

m represents an integer from 2 to 10; and

n represents 0 or 1.

The PNA oligomer employed in the present invention may be synthesized according to the method of Korean Patent No. 464261, the entire contents of which is incorporated herein by reference, by using PNA monomers protected with Bts (benzothiazolesulfonyl), Fmoc (9-flourenylmethloxycarbonyl) or t-Boc (t-butoxycarbonyl) group (see J. Org. Chem. 59, 5767-5773, J Peptide Sci. 3, 175-183, Tetrahedron Lett. 22, 6179-6194, and International Publication No. WO 2008/072933), the entire contents of which is incorporated herein by reference.

3. Manufacture of PNA Chip

The probes designed in the above 2. are immobilized on a support(s) of silica, semi-conductor, plastic, gold, silver, magnetic molecules, or polymer such as nylon, poly(dimethylsiloxane) (PDMS), cellulose and nitrocellulose, particularly, glass slide. The form of the support is not particularly limited, but it may be, for example, a hand holdable thin plate such as a glass slide, a tube, or a bead having the diameter of 0.1 mm or less which can be transferred in admixture with liquid. Further, a multi-well plate, particularly a 96-well plate, onto which functional groups are attached, may be used. The surface of the support may be functionalized with a functional group such as aldehyde, carboxyl, epoxy, isothiocyanate, N-hydroxysuccinimidyl or activated ester group, particularly, with epoxy group.

Upon immobilization of the probes, the functional groups such as residual amine or epoxy group may be blocked and treated to reduce the background signal (see Example 3).

The kit for expression profiling of miRNAs according to the present invention may be utilized for various analysis, diagnosis, or the like. For example, it may be used for tumor subtyping or prognosis.

4. Establishment of Conditions for Reaction and Analysis on a PNA Chip

The method for expression profiling of miRNAs according to the present invention may comprise:

(a) extracting RNA, which is a target of the PNA chip;

(b) optionally, labeling the target miRNA with a fluorescent dye;

(c) performing hybridization between probe PNA and the target miRNA;

(d) washing to remove residual reactants following the hybridization;

(e) optionally, attaching a detectable label to the hybridized miRNA;

(f) washing to remove residual reactants; and

(g) detecting a signal from the hybridization.

In step (a), any method to extract RNA commonly used in the art may be used. The

RNA extraction method is not specially limited because no special process for isolation of RNA is required. For example, RNA may be extracted from blood or specific tissues using Trizol, or a commercialized product.

Further, PAGE fractionation (flashPAGE fractionator), which is used for isolation of short RNA or miRNA, may also be used.

The samples useful for the present invention may be obtained from various sources. For example, they may be obtained from different individuals or from different developmental stages of one individual.

In step (b), the method for labeling miRNA from the total RNA with a fluorescent dye is not particularly limited. A variety of commercialized labeling kits may be used. Representative examples thereof include: attachment of a fluorophore at the 5′ terminal of miRNA using T4 polynucleotide kinase (Agilent Inc.); and attachment of fluorophore-labeled RNA linker using T4 RNA ligase (see the literature [Castoldi M et al., 2007, Method. 43:146-152]). Further, poly(A) polymerase may be used to attach fluorophore-labeled poly(A) at the end of miRNA. Besides, a fluorescent dye may be attached through chemical or other various methods (see the literature [Enos M et al., 2007, Biotechniques. 42(3)]). If miRNA is labeled with fluorophore in step (b), step (e) is omitted.

In step (c), hybridization is carried out. The target is mixed with a suitable hybridization buffer and reacted at an appropriate temperature so that the probes bind to complementary target miRNAs. It is preferable to use an appropriate hybridization buffer to facilitate the hybridization.

In step (d), washing is carried out. The unreacted reactants such as residual target nucleic acids are removed so that only the target RNAs complementarily bound to the probes remain.

In step (e), the target nucleic acids are labeled with a fluorescent dye for detection (so called post-labeling, see the co-pending Korean Patent Application No. 10-2008-0120122, the entire contents of which is incorporated herein by reference). In case of carrying out step (e), the target miRNA is not labeled with a fluorescent dye in step (b). Following the hybridization, only the target miRNAs bound to the immobilized PNA probes are labeled with a fluorescent dye. Because the PNA employed in the present invention is very stable against nucleases or other biological enzymes, fluorescent labeling occurs only in the miRNAs complementarily bound to the PNA probes, but not in the PNA probes. For instance, a fluorophore is attached to the single-stranded miRNA using such an enzyme as terminal deoxynucleotidyl transferase, T4 RNA ligase, etc. T4 RNA ligase is an enzyme that adds nucleotides at the 5′ terminal. Preferably, it attaches ddNTP or RNA linker, or various fluorophore-labeled linkers at the 5′ terminal of RNA. A fluorophore may be directly attached to dNTP, ddNTP or bisphosphate linker (see the literature [Wang et al., 2007, RNA. 13:151-159]), or a substance reactive with a fluorophore such as biotin may be used. Specifically, a fluorophore such as Cy5 or Cy3 may be directly attached to dNTP, e.g. dCTP, or a substance reactive with a fluorophore such as biotin may be used. ddNTP may also be used, and an oligonucleotide including a fluorophore may also be used.

Further, a chemical may be used to attach a fluorophore. The chemical may be one selectively labeling the miRNA which is hybridized with the PNA probe, without reacting with the PNA probe. It may label the miRNA at its end or internal region. Labels that can be employed in the present invention are not particularly limited. For example, biotin, rhodamine, cyanine 3, cyanine 5, pyrene, cyanine 2, green fluorescent protein (GFP), calcein, fluorescein isothiocyanate (FITC), Alexa 488, 6-carboxy-fluorescein (FAM), 2′,4′,5′,7′-tetrachloro-6-carboxy-4,7-dichlorofluorescein (HEX), 2′,7′-dichloro-6-carboxy-4,7-dichlorofluorescein (TET), fluorescein chloro-triazinyl, fluorescein, Oregon Green, Magnesium Green, Calcium Green, 6-carboxy-4′,5′-dichloro-2′,7′-dimethoxyfluorescein (JOE), tetramethylrhodamine, tetramethylrhodamine isothiocyanate (TRITC), carboxytetramethylrhodamine (TAMRA), rhodamine phalloidin, pyronin Y, Lissamine, X-rhodamine (ROX), Calcium Crimson, Texas Red, Nile Red and thiadicarbocyanine may be used.

The method of selectively labeling the target nucleic acid with a fluorescent dye after hybridization in accordance with the present invention could hardly be applied to a DNA chip. This is because in addition to the target miRNAs, the DNA probes immobilized on the support also react with terminal deoxynucleotidyl transferase and are labeled with the fluorescent dye, making it difficult to distinguish them from the miRNAs hybridized therewith. For this reason, when a DNA chip is used, the extracted RNA is labeled with a fluorescent dye. A large amount of fluorescent dye and enzyme are required to attach the fluorescent dye to all the RNA fragments. In contrast, the post-hybridization labeling of the target nucleic acid bound to the probe with a fluorescent dye using terminal deoxynucleotidyl transferase in accordance with the present invention involves the reduced number of steps with omitting the pre-treatment step of eliminating the residual reactants, to require less effort and time. Further, the labeling may be accomplished more efficiently with a much smaller amount of enzyme and fluorescent dye, as compared to the labeling of total extracted RNAs.

In step (f), washing is carried out to remove unreacted residual label and enzyme.

In step (g), nucleic acid hybridization is detected. Any methods for detecting hybridization may be employed, including fluorescence detection, electrochemical detection, and detection based on change in mass, charge, or optical properties. Further, detection may be carried out through antibody-antigen reactions using a chemiluminescent compound or enzyme. For example, it may be carried out by enzymatic colorimetry using streptavidin (STR) which binds to biotin and horseradish peroxidase (HRP). In a specific embodiment, fluorescence emitted from the binding of biotin with streptavidin-Cy5 or -Cy3 may be detected.

### EXAMPLES

Hereinafter, the present invention will be explained in more detail with reference to specific examples. However, the present invention is not limited by those examples in any manner, and it would be apparent to those skilled in the art that various alterations and modifications can be made within the spirit and scope of the present invention.

### Example 1

### Synthesis of PNA Oligomers for Expression Profiling of miRNAs

One-hundred-forty-four (144) PNA probes for expression profiling of miRNAs were prepared to have base sequences given in Table 1. Each probe was synthesized to have multi-amine linkers at the N- and C-terminals for immobilization on a glass slide (see the literatures [J. Org. Chem. 59, 5767-5773], [J. Peptide Sci. 3, 175-183] and [Tetrahedron Lett. 22, 6179-6194], and International Publication No. WO03/091231).

### Example 2

### Preparation of Target miRNAs and Labeling with Fluorescent Dyes

Synthetic RNAs having the identical base sequences to those of the target miRNAs were used to investigate the complementary binding characteristic and sensitivity of the probes. The RNA was synthesized by Bioneer (Korea). The synthesized RNA had biotin attached at its 5′ terminal, and was labeled with a fluorescent dye using Label IT miRNA labeling kit (Miurs Inc.).

### Example 3

### Manufacture of a PNA Chip

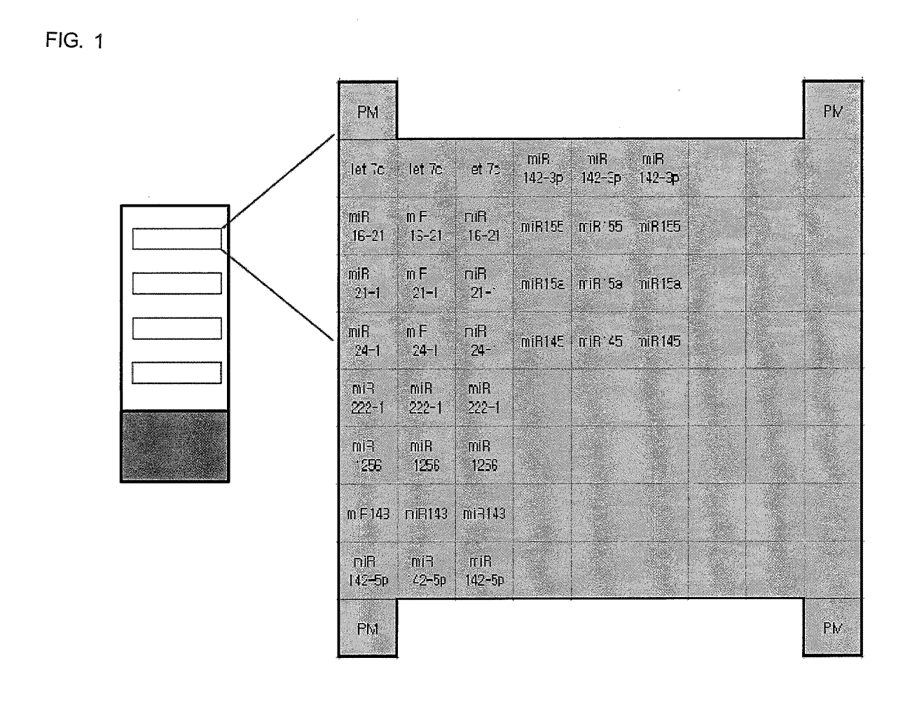

The purified PNA oligomers as shown in Table 1 were diluted with a spotting buffer to 50 uM. They were spotted on a glass slide functionalized with epoxy group by pin-spotting method, and the slide was allowed to stand at room temperature with 75% humidity for 4 hours. Then, it was added to dimethylformamide (DMF) and washed with ultrasonication for 15 minutes. It was added to DMF supplemented with 0.1 M succinic anhydride, and the unreacted amine group was removed at 40 C for 2 hours. Upon completion of the reaction, the reaction solution was removed, and the slide was washed sequentially with DMF and triple distilled water, with ultrasonication for 15 minutes. Upon completion of the reaction, 100 mM Tris-HCl buffer containing 0.1 M ethanolamine was added thereto to inactivate the residual epoxy group on the surface of the slide. The glass slide was further washed twice with triple distilled water with ultrasonication for 15 minutes, treated with boiling water for 5 minutes, washed with triple distilled water for 5 minutes, and then dried. Then, a silicon reactor capable of containing 100 L of hybridization solution was attached onto the glass slide. FIG. 1 schematically shows the PNA chip according to an embodiment of the present invention.

### Example 4

### Hybridization with miRNAs

5 L of fragmented PCR product was added to 100 L of PNAArray hybridization buffer (Panagene, Korea). The hybridization mixture (100 L) was injected on a glass slide, and reaction was performed at 40 C for 2 hours.

Upon completion of the reaction, the reaction mixture was washed with PNAArray washing buffer (Panagene) twice at room temperature for 5 minutes, and then dried.

Using a fluorescence scanner (GenePix 4000B, US), the glass slide was imaged.

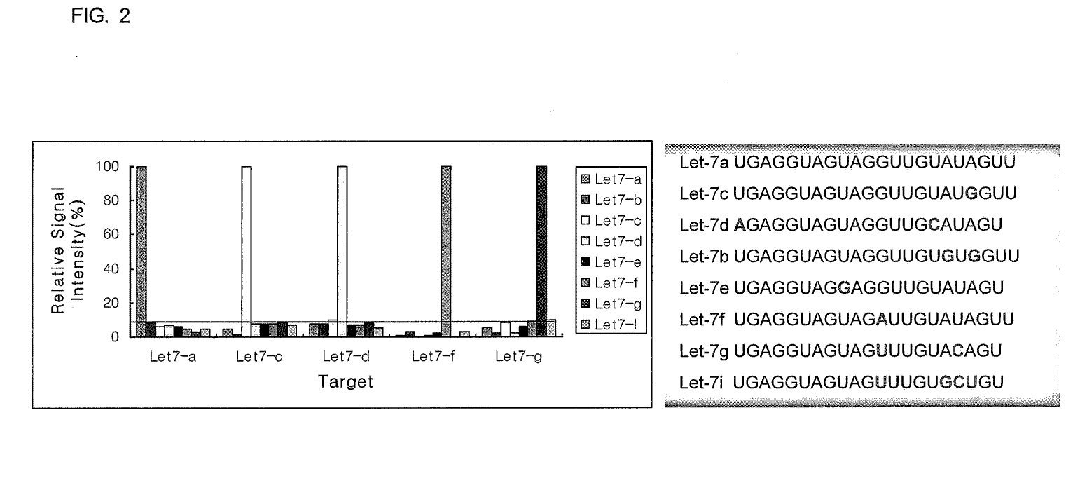

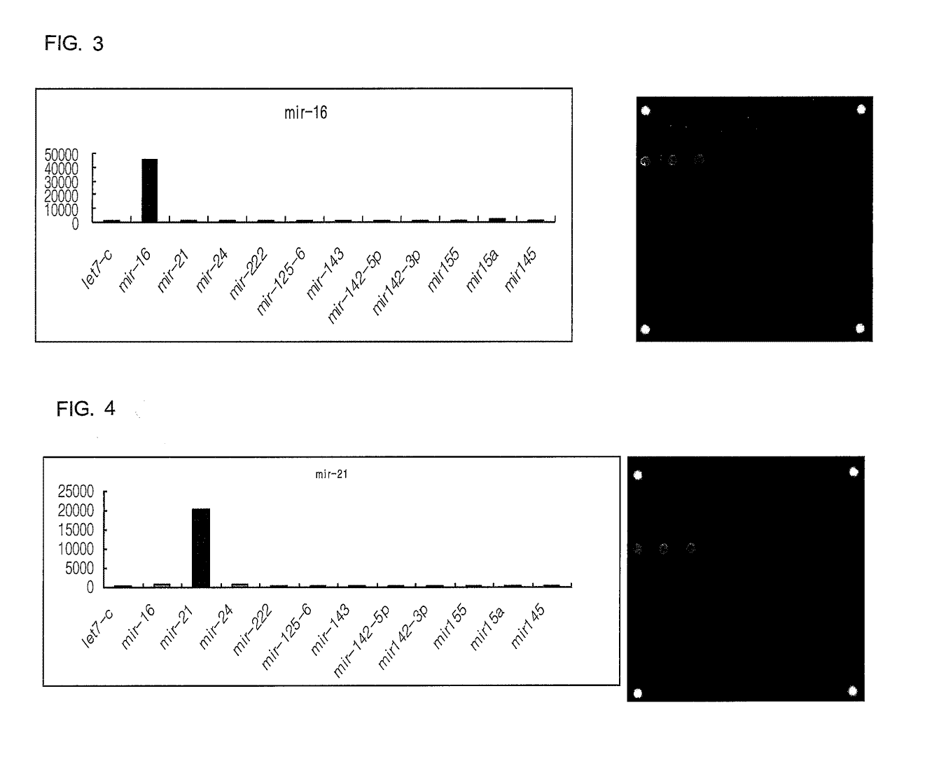

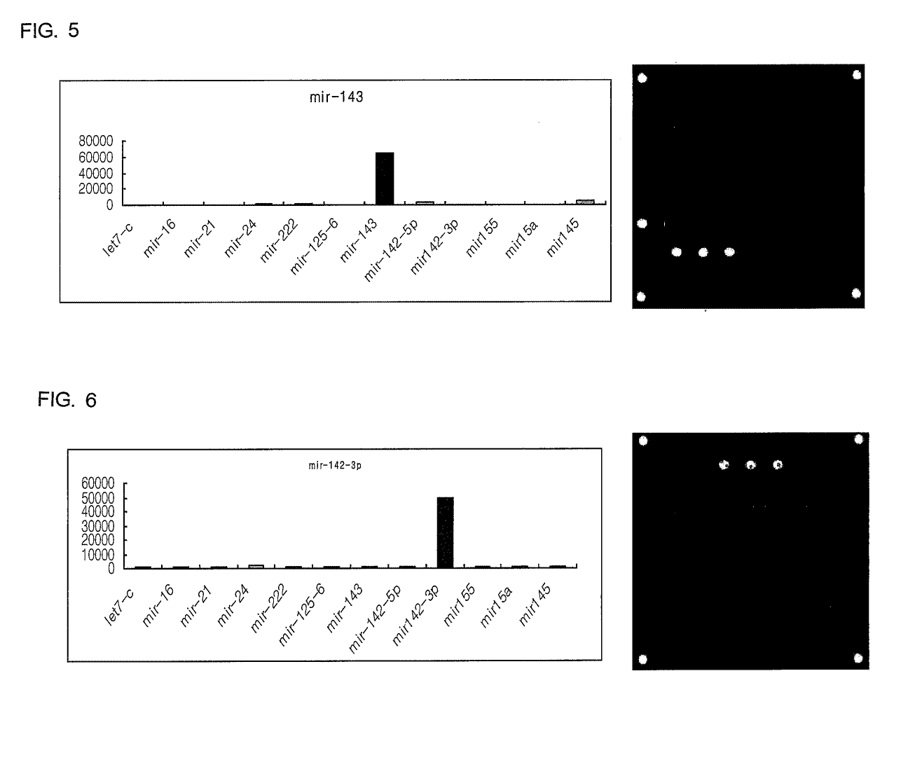

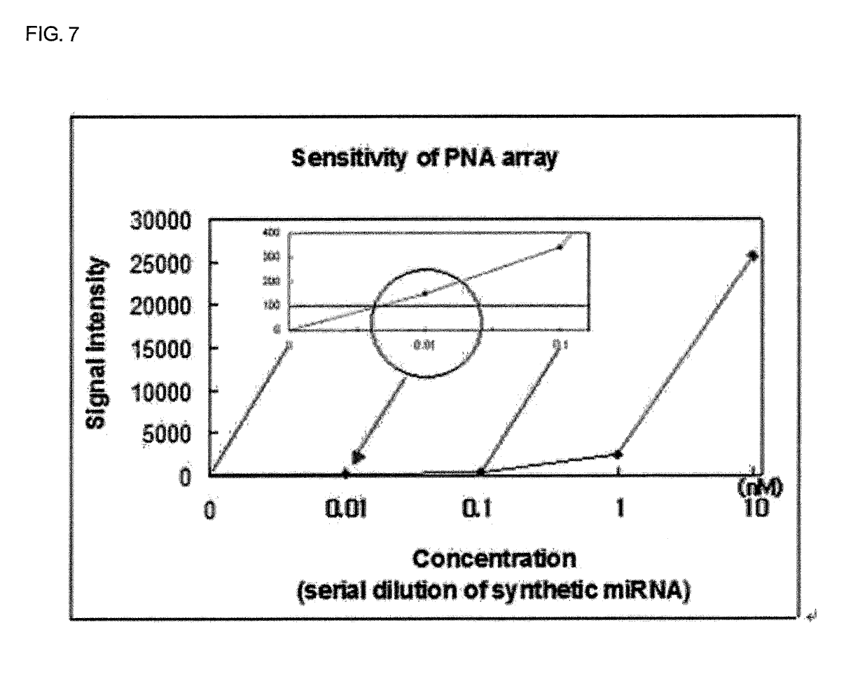

The result is shown in FIGS. 2 to 7. As shown in FIG. 2, a high discrimination could be obtained even with a single nucleotide difference in let-7 family (let-7a to let-7g). As shown in FIGS. 3 to 6, for each miRNA target, a specific signal from the miRNA specifically hybridized was obtained, and the specific signal could be discriminated from the non-specific one. In addition, as shown in FIG. 7, the chip according to the present invention could detect even a low concentration of miRNA target, showing a high sensitivity.

### SEQUENCE LISTING

SEQ ID No. 1 represents the nucleotide sequence of probe let-7a;

SEQ ID No. 2 represents the nucleotide sequence of probe let-7b;

SEQ ID No. 3 represents the nucleotide sequence of probe let-7c;

SEQ ID No. 4 represents the nucleotide sequence of probe let-7d;

SEQ ID No. 5 represents the nucleotide sequence of probe let-7e;

SEQ ID No. 6 represents the nucleotide sequence of probe let-7f;

SEQ ID No. 7 represents the nucleotide sequence of probe let-7g;

SEQ ID No. 8 represents the nucleotide sequence of probe miR-16;

SEQ ID No. 9 represents the nucleotide sequence of probe miR-21;

SEQ ID No. 10 represents the nucleotide sequence of probe miR-24;

SEQ ID No. 11 represents the nucleotide sequence of probe miR-222;

SEQ ID No. 12 represents the nucleotide sequence of probe miR-125b;

SEQ ID No. 13 represents the nucleotide sequence of probe miR-143;

SEQ ID No. 14 represents the nucleotide sequence of probe miR-142-5p;

SEQ ID No. 15 represents the nucleotide sequence of probe miR-142-3p;

SEQ ID No. 16 represents the nucleotide sequence of probe miR-155;

SEQ ID No. 17 represents the nucleotide sequence of probe miR-15a;

SEQ ID No. 18 represents the nucleotide sequence of probe miR-145;

SEQ ID No. 19 represents the nucleotide sequence of probe miR-196a-1;

SEQ ID No. 20 represents the nucleotide sequence of probe miR-196a-2;

SEQ ID No. 21 represents the nucleotide sequence of probe miR-196b-1;

SEQ ID No. 22 represents the nucleotide sequence of probe miR-196b-2;

SEQ ID No. 23 represents the nucleotide sequence of probe miR-196b-3;

SEQ ID No. 24 represents the nucleotide sequence of probe miR-19a-1;

SEQ ID No. 25 represents the nucleotide sequence of probe miR-19a-2;

SEQ ID No. 26 represents the nucleotide sequence of probe miR-19b-1;

SEQ ID No. 27 represents the nucleotide sequence of probe miR-19b-2;

SEQ ID No. 28 represents the nucleotide sequence of probe miR-221-1;

SEQ ID No. 29 represents the nucleotide sequence of probe miR-221-2;

SEQ ID No. 30 represents the nucleotide sequence of probe miR-221-3;

SEQ ID No. 31 represents the nucleotide sequence of probe miR-181a;

SEQ ID No. 32 represents the nucleotide sequence of probe miR-181b;

SEQ ID No. 33 represents the nucleotide sequence of probe miR-181c;

SEQ ID No. 34 represents the nucleotide sequence of probe miR-18;

SEQ ID No. 35 represents the nucleotide sequence of probe miR-224;

SEQ ID No. 36 represents the nucleotide sequence of probe miR-199b;

SEQ ID No. 37 represents the nucleotide sequence of probe miR-195;

SEQ ID No. 38 represents the nucleotide sequence of probe miR-200a;

SEQ ID No. 39 represents the nucleotide sequence of probe miR-146a;

SEQ ID No. 40 represents the nucleotide sequence of probe miR-372;

SEQ ID No. 41 represents the nucleotide sequence of probe miR-373;

SEQ ID No. 42 represents the nucleotide sequence of probe miR-20a;

SEQ ID No. 43 represents the nucleotide sequence of probe miR-21;

SEQ ID No. 44 represents the nucleotide sequence of probe miR-22;

SEQ ID No. 45 represents the nucleotide sequence of probe miR-189;

SEQ ID No. 46 represents the nucleotide sequence of probe miR-29b.

SEQ ID No. 47 represents the nucleotide sequence of probe miR-let7i.

SEQ ID No. 48 represents the nucleotide sequence of probe miR-1.

SEQ ID No. 49 represents the nucleotide sequence of probe miR-100.

SEQ ID No. 50 represents the nucleotide sequence of probe miR-101.

SEQ ID No. 51 represents the nucleotide sequence of probe miR-103.

SEQ ID No. 52 represents the nucleotide sequence of probe miR-106a.

SEQ ID No. 53 represents the nucleotide sequence of probe miR-106b.

SEQ ID No. 54 represents the nucleotide sequence of probe miR-107.

SEQ ID No. 55 represents the nucleotide sequence of probe miR-10a.

SEQ ID No. 56 represents the nucleotide sequence of probe miR-10b.

SEQ ID No. 57 represents the nucleotide sequence of probe miR-122.

SEQ ID No. 58 represents the nucleotide sequence of probe miR-124a.

SEQ ID No. 59 represents the nucleotide sequence of probe miR-125a.

SEQ ID No. 60 represents the nucleotide sequence of probe miR-126.

SEQ ID No. 61 represents the nucleotide sequence of probe miR-127-3p.

SEQ ID No. 62 represents the nucleotide sequence of probe miR-127-5p.

SEQ ID No. 63 represents the nucleotide sequence of probe miR-128.

SEQ ID No. 64 represents the nucleotide sequence of probe miR-132.

SEQ ID No. 65 represents the nucleotide sequence of probe miR-133a.

SEQ ID No. 66 represents the nucleotide sequence of probe miR-133b.

SEQ ID No. 67 represents the nucleotide sequence of probe miR-134.

SEQ ID No. 68 represents the nucleotide sequence of probe miR-135a.

SEQ ID No. 69 represents the nucleotide sequence of probe miR-135b.

SEQ ID No. 70 represents the nucleotide sequence of probe miR-136.

SEQ ID No. 71 represents the nucleotide sequence of probe miR-137.

SEQ ID No. 72 represents the nucleotide sequence of probe miR-140-3p.

SEQ ID No. 73 represents the nucleotide sequence of probe miR-140-5p.

SEQ ID No. 74 represents the nucleotide sequence of probe miR-141.

SEQ ID No. 75 represents the nucleotide sequence of probe miR-146b.

SEQ ID No. 76 represents the nucleotide sequence of probe miR-148a.

SEQ ID No. 77 represents the nucleotide sequence of probe miR-149.

SEQ ID No. 78 represents the nucleotide sequence of probe miR-150.

SEQ ID No. 79 represents the nucleotide sequence of probe miR-151.

SEQ ID No. 80 represents the nucleotide sequence of probe miR-153.

SEQ ID No. 81 represents the nucleotide sequence of probe miR-154.

SEQ ID No. 82 represents the nucleotide sequence of probe miR-15b.

SEQ ID No. 83 represents the nucleotide sequence of probe miR-17-3p.

SEQ ID No. 84 represents the nucleotide sequence of probe miR-17-5p.

SEQ ID No. 85 represents the nucleotide sequence of probe miR-181d.

SEQ ID No. 86 represents the nucleotide sequence of probe miR-182.

SEQ ID No. 87 represents the nucleotide sequence of probe miR-183.

SEQ ID No. 88 represents the nucleotide sequence of probe miR-185.

SEQ ID No. 89 represents the nucleotide sequence of probe miR-186.

SEQ ID No. 90 represents the nucleotide sequence of probe miR-188-3p.

SEQ ID No. 91 represents the nucleotide sequence of probe miR-188-5p.

SEQ ID No. 92 represents the nucleotide sequence of probe miR-18b.

SEQ ID No. 93 represents the nucleotide sequence of probe miR-190b.

SEQ ID No. 94 represents the nucleotide sequence of probe miR-191.

SEQ ID No. 95 represents the nucleotide sequence of probe miR-192.

SEQ ID No. 96 represents the nucleotide sequence of probe miR-194.

SEQ ID No. 97 represents the nucleotide sequence of probe miR-197.

SEQ ID No. 98 represents the nucleotide sequence of probe miR-198.

SEQ ID No. 99 represents the nucleotide sequence of probe miR-199a.

SEQ ID No. 100 represents the nucleotide sequence of probe miR-199a-3p.

SEQ ID No. 101 represents the nucleotide sequence of probe miR-200b.

SEQ ID No. 102 represents the nucleotide sequence of probe miR-200c.

SEQ ID No. 103 represents the nucleotide sequence of probe miR-202.

SEQ ID No. 104 represents the nucleotide sequence of probe miR-203.

SEQ ID No. 105 represents the nucleotide sequence of probe miR-204.

SEQ ID No. 106 represents the nucleotide sequence of probe miR-205.

SEQ ID No. 107 represents the nucleotide sequence of probe miR-206.

SEQ ID No. 108 represents the nucleotide sequence of probe miR-210.

SEQ ID No. 109 represents the nucleotide sequence of probe miR-214.

SEQ ID No. 110 represents the nucleotide sequence of probe miR-215.

SEQ ID No. 111 represents the nucleotide sequence of probe miR-216a.

SEQ ID No. 112 represents the nucleotide sequence of probe miR-216b.

SEQ ID No. 113 represents the nucleotide sequence of probe miR-218.

SEQ ID No. 114 represents the nucleotide sequence of probe miR-219.

SEQ ID No. 115 represents the nucleotide sequence of probe miR-223.

SEQ ID No. 116 represents the nucleotide sequence of probe miR-23a.

SEQ ID No. 117 represents the nucleotide sequence of probe miR-25.

SEQ ID No. 118 represents the nucleotide sequence of probe miR-26a.

SEQ ID No. 119 represents the nucleotide sequence of probe miR-26b.

SEQ ID No. 120 represents the nucleotide sequence of probe miR-27a.

SEQ ID No. 121 represents the nucleotide sequence of probe miR-27b.

SEQ ID No. 122 represents the nucleotide sequence of probe miR-28-5p.

SEQ ID No. 123 represents the nucleotide sequence of probe miR-296-3p.

SEQ ID No. 124 represents the nucleotide sequence of probe miR-296-5p.

SEQ ID No. 125 represents the nucleotide sequence of probe miR-29a.

SEQ ID No. 126 represents the nucleotide sequence of probe miR-29c.

SEQ ID No. 127 represents the nucleotide sequence of probe miR-30a.

SEQ ID No. 128 represents the nucleotide sequence of probe miR-30b.

SEQ ID No. 129 represents the nucleotide sequence of probe miR-30C.

SEQ ID No. 130 represents the nucleotide sequence of probe miR-31.

SEQ ID No. 131 represents the nucleotide sequence of probe miR-342-3p.

SEQ ID No. 132 represents the nucleotide sequence of probe miR-342-5p.

SEQ ID No. 133 represents the nucleotide sequence of probe miR-34a.

SEQ ID No. 134 represents the nucleotide sequence of probe miR-368.

SEQ ID No. 135 represents the nucleotide sequence of probe miR-375.

SEQ ID No. 136 represents the nucleotide sequence of probe miR-488.

SEQ ID No. 137 represents the nucleotide sequence of probe miR-7.

SEQ ID No. 138 represents the nucleotide sequence of probe miR-9.

SEQ ID No. 139 represents the nucleotide sequence of probe miR-9*.

SEQ ID No. 140 represents the nucleotide sequence of probe miR-92a.

SEQ ID No. 141 represents the nucleotide sequence of probe miR-92b.

SEQ ID No. 142 represents the nucleotide sequence of probe miR-93.

SEQ ID No. 143 represents the nucleotide sequence of probe miR-95.

SEQ ID No. 144 represents the nucleotide sequence of probe miR-99a.

1. A peptide nucleic acid (PNA) probe capable of specifically binding to a target microRNA (miRNA), which consists of 13 to 22 bases and includes base sequences complementary to 3 to 10 base sequences in 5′ seed of the target miRNA.

2. The PNA probe according to claim 1, wherein the target miRNA is selected from the group consisting of let-7a, let-7b, let-7c, let-7d, let-7e, let-7f, let-7g, let-7i, miR-1, miR-1b, miR-1d, miR-2, miR-7, miR-7b, miR-9, miR-9*, miR-10a, miR-10b, miR-12a, miR-15a, miR-15b, miR-16, miR-16-1, miR-17-3p, miR-17-5p, miR-18, miR-18b, miR-19a, miR-19b, miR-20a, miR-21, miR-22, miR-23a, miR-23b, miR-24, miR-25,miR-26a, miR-26b, miR-27a, miR-28-5p, miR-29, miR-29b, miR-29c, miR-31, miR-34, miR-34a, miR-92a, miR-92b, miR-93, miR-95, miR-99a, miR-100, miR-101, miR-102, miR-103, miR-106a, miR-106b, miR-107, miR-122, miR-124, miR-124b, miR-125a, miR-125b, miR-126, miR-127, miR-128, miR-132, miR-133a, miR-133b, miR-134, miR-135a, miR-135b, miR-136, miR-137, miR-140-3p, miR-141, miR-142-5p, miR-142-3p, miR-143, miR-145, miR-146a, miR-146b, miR-148a, miR-149, miR-150, miR-151, miR-153, miR-154, miR-155, miR-181, miR-181a, miR-181b, miR-181c, miR-181d, miR-182, miR-183, miR-184, miR-185, miR-186, miR-188-3p, miR-188-5p, miR-189, miR-190b, miR-191, miR-192, miR-194, miR-195, miR-196a, miR-196b, miR-197, miR-198, miR-199a, miR-199a-3p, miR-199b, miR-200a, miR-200b, miR-200c, miR-202, miR-203, miR-204, miR-205, miR-206, miR-208, miR-210, miR-211, miR-212, miR-213, miR-214, miR-215, miR-216a, miR-216b, miR-218, miR-219, miR-221, miR-222, miR-223, miR-224, miR-296, miR-301, miR-342-3p, miR-342-5p, miR-363, miR-368, miR-372, miR-373, miR-375, miR-376, miR-380 miR-430, and miR-488.

3. The PNA probe according to claim 2, which consists of any one of nucleotide sequences as set forth in SEQ ID Nos. 1 to 144.

4. A kit for expression profiling of miRNA, which comprises a support(s) and one or more of the PNA probe(s) according to claim 1, the PNA probe(s) being immobilized on the support(s).

5. The kit according to claim 4, for use in tumor subtyping or prognosis.

6. The kit according to claim 4, wherein the support is selected from the group consisting of glass slide, silica, semiconductor, plastic, gold, silver, magnetic molecules, nylon, polydimethylsiloxane (PDMS), cellulose and nitrocellulose.

7. The kit according to claim 4, wherein the support has the form of thin plate, tube or bead.

8. The kit according to claim 4, wherein the support is a multi-well plate.

9. A method for expression profiling of miRNA, which comprises:
introducing a reaction sample containing miRNA to the kit including the PNA probe(s) according to claim 4;
performing hybridization reaction between the PNA probe(s) and the miRNA; and
detecting a signal from the hybridization.

10. The method according to claim 9, wherein, after the hybridization, a detectable label and an agent capable of introducing the label to the miRNA are added, so that the miRNA is reacted with the agent to be selectively labeled with the label.

11. The method according to claim 10, wherein the agent capable of introducing the detectable label is an enzyme introducing the detectable label at the end of the miRNA, or a chemical introducing the detectable label at the end or internal region of the miRNA.

12. The method according to claim 11, wherein the enzyme introducing the detectable label at the end of the miRNA is terminal deoxynucleotidyl transferase or ligase.

13. The method according to claim 12, wherein the detectable label is attached to ddNTP, dNTP or RNA linker.

14. The method according to claim 13, wherein the detectable label is detected through antibody-antigen reaction using a chemiluminescent compound or enzyme.
